(19) United States
(12) Patent Application Publication
(10) Pub. No.: US 2025/0161181 A1
(43) Pub. Date: May 22, 2025

(54) Personal Care Compositions

(71) Applicant: Colgate-Palmolive Company, New York, NY (US)

(72) Inventors: Yanjun DENG, Guangzhou (CN); Yuyan ZENG, Guangzhou (CN); Dongyao HUANG, Guangzhou (CN)

(73) Assignee: Colgate-Palmolive Company, New York, NY (US)

(21) Appl. No.: 18/951,172

(22) Filed: Nov. 18, 2024

(30) Foreign Application Priority Data
Nov. 21, 2023 (CN) 202311564110.6

(51) Int. Cl. A61K 8/46; A61K 8/34; A61K 8/37; A61K 8/44; A61Q 19/00

(52) U.S. Cl. CPC A61K 8/466; A61K 8/345; A61K 8/375; A61K 8/44; A61K 8/463; A61Q 19/00; A61K 2800/10; A61K 2800/30

(57) ABSTRACT

Personal care compositions are disclosed herein. In accordance with an aspect, provided is a personal care composition comprising: (a) a surfactant system comprising at least one anionic surfactant and at least one amphoteric surfactant; and (b) at least one tear film protecting agent, wherein the personal care composition has a pH ranging from about 4.0 to about 6.0.

### CROSS-REFERENCE TO RELATED APPLICATION

This application claims the benefit of priority from Chinese Application No. 202311564110.6, filed 21 Nov. 2023, the contents of which are hereby incorporated herein by reference in their entirety.

### BACKGROUND

Personal care composition products like body washes, shampoos, conditioners, and shower gels are becoming more popular in the United States and around the world. Desirable personal care compositions must meet a number of criteria. For example, in order to be acceptable to consumers, a body wash composition should exhibit good cleaning properties, should exhibit good lathering characteristics, should be mild to the skin (not cause drying or irritation), and preferably should even provide a conditioning benefit to the skin.

Body washes (sometimes referred to as shower gels) are a general term used to describe liquid surfactant containing formulations used to clean the body, while shampoos and conditioners are general terms to describe liquid surfactant containing formulations used to claims and/or condition the hair. Frequently, a personal care composition may serve to clean and/or condition the body, including both skin and hair. While sometimes considered generically as “soap,” such personal care compositions frequently do not contain soaps such as sodium or potassium salts of fatty acids. Conventional personal care compositions typically contain one of a variety of components including one or more surfactants, various emollients, fragrances and other personal care ingredients. Conventional personal care compositions offer less skin irritation, improved lather in hard water conditions and leave less residues on the skin and bathroom fixtures when compared with common soaps.

Typical personal care compositions are generally intended for adult use. Young children, including infants and toddlers, may have more delicate, sensitive skin and may be more sensitive to eye irritation resulting personal care compositions incidentally being deposited into the eye as a result of the body and/or hair washing process. Many conventional personal care compositions may therefore be unsuitable for use with children due to harsh ingredients. Accordingly, gentle, environmentally-acceptable personal care compositions for young children are desirable, such as products designated as “tear free” body wash and/or shampoo formulations and/or products containing mostly natural or all natural ingredients, including ingredients that are biodegradable.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

In accordance with an aspect, provided is a personal care composition comprising (a) a surfactant system comprising at least one anionic surfactant and at least one amphoteric surfactant; and (b) at least one tear film protecting agent, wherein the personal care composition has a pH ranging from about 4.0 to about 6.0, such as a pH ranging from about 4.2 to about 6.8 or from about 4.2 to about 5.2. In certain embodiments, the personal care composition may have a natural origin index of about 90% or more, such as about 96% or more or about 97% or more, based on ISO Standard 16128.

In accordance with certain aspects, the at least one anionic surfactant of the personal care composition disclosed herein is selected from sulfate anionic surfactants, such as sodium laureth sulfate (SLES). In accordance with certain other aspects, the at least one anionic surfactant is selected from sulfolaurate surfactants, such as disodium-2-sulfolaurate, and in certain embodiments, the personal care composition is free of or substantially free of sulfate anionic surfactants such as SLES. In certain embodiments, the at least one anionic surfactant is present in the personal care composition in an amount ranging from about 3 to about 10 wt. %, based on the total weight of the personal care composition.

In accordance with certain aspects, the at least one amphoteric surfactant of the personal care composition disclosed herein is selected from betaine surfactants, such as cocamidopropyl betaine, optionally present in an amount ranging from about 5 to about 20 wt. %, based on the total weight of the personal care composition.

In accordance with certain aspects, the surfactant system of the personal care composition disclosed herein further comprises at least one non-ionic surfactant, such as a glucoside surfactant, preferably cocoglucoside.

In accordance with certain aspects, the at least one tear film protecting agent of the personal care composition disclosed herein is selected from glycerin and glyceryl oleate, optionally present in an amount ranging from about 4 to about 8 wt. %, based on the total weight of the personal care composition.

In according with certain aspects, the surfactant system of the personal care composition disclosed herein further comprises a taurate surfactant, preferably sodium methyl oleyl taurate. In certain aspects, the personal care composition further comprises at least one preservative, such as a preservative selected from sodium benzoate and potassium sorbate, and in certain aspects, the personal care composition further comprises at least one anti-allergy agent, such as dipropylene glycol (DPG) or bisabolol. In certain aspects, the personal care composition disclosed herein is free of thickeners, e.g., thickeners that are not otherwise part of the surfactant system.

In accordance with another aspect, provided is a personal care composition comprising (a) a surfactant system comprising disodium-2-sulfolaurate, a glucoside surfactant, and a betaine surfactant; and (b) at least two tear film protecting agents comprising glycerin and glyceryl oleate, wherein the personal care composition is free of sulfate anionic surfactants and has a pH ranging from about 4.0 to about 6.0. In accordance with another aspect, provided is a personal care composition comprising (a) a surfactant system comprising a sulfate anionic surfactant and a betaine surfactant; and (b) at least two tear film protecting agents comprising glycerin and glyceryl oleate, wherein the personal care composition has a pH ranging from about 4.0 to about 6.0.

### DETAILED DESCRIPTION

For illustrative purposes, the principles of the present disclosure are described by referencing various exemplary embodiments thereof. Although certain embodiments of the disclosure are specifically described herein, one of ordinary skill in the art will readily recognize that the same principles are equally applicable to and can be employed in other compositions and methods. Before explaining the disclosed embodiments of the present disclosure in detail, it is to be understood that the disclosure is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to.” The term “including” should be interpreted as “including, but are not limited to.”

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “^{∘}” refers to a degree, such as a temperature degree or a degree of an angle. The symbols “h”, “min”, “mL”, “nm”, and “μm” refer to hour, minute, milliliter, nanometer, and micrometer, respectively. The abbreviation “rpm” means revolutions per minute.

When referring to chemical structures, and names, the symbols “C”, “H”, and “O” mean carbon, hydrogen, and oxygen, respectively. The symbols “-”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the present disclosure can be free or essentially free of all components and elements positively recited throughout the present disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds may be characterized as both a surfactant and an emulsifier. If a particular personal care composition includes both a surfactant and an emulsifier, a compound that may be characterized as both a surfactant and an emulsifier will serve only as either a surfactant or an emulsifier—not both.

For readability purposes, the chemical functional groups are in their adjective form; for each of the adjectives, the word “group” is assumed. For example, the adjective “alkyl” without a noun thereafter, should be read as “an alkyl group.”

Certain aspects of the disclosure are directed to personal care compositions and personal care compositions preferably having a natural origin index of about 90% or more, e.g., as measured pursuant to ISO Standard 16128. It has been discovered that personal care compositions formulated with certain surfactant systems can advantageously achieve desirable thickening while simultaneously having a preferable natural origin index and/or a biodegradability index and also being gentle enough for use on a young child or baby's delicate skin and without causing undesirable eye irritation. ISO Standard 16128-1:2016 provides guidelines for the definitions of natural and organic cosmetic ingredients.

The personal care compositions may have a natural origin index of about 90% or more, e.g., as measured pursuant to ISO Standard 16128. For example, in some embodiments, the personal care composition may have a natural origin index pursuant to ISO Standard 16128 of about 90% or more, about 91% or more, about 92% or more, about 93% or more, about 94% or more, about 95% or more, about 96% or more, about 97% or more, about 98% or more, about 99% or more, or about 99.5% or more.

The personal care compositions may, additionally or alternatively, achieve a biodegradability index of about 85% or more, e.g. as measured pursuant to ISO Standard 14855-1:2005. In some instances, the personal care compositions may have a biodegradability index, e.g., pursuant to ISO Standard 14855-1:2005, of about 85% or more, about 87% or more, about 88% or more, about 89% or more, about 90% or more, about 91% or more, about 92% or more, about 93% or more, about 94% or more, about 95% or more, about 96% or more, about 97% or more, about 98% or more, about 99% or more, or about 99.5% or more.

In accordance with an aspect, provided is a personal care composition comprising either a sulfate-based surfactant system or a sulfate-free surfactant system. The personal care compositions according to certain embodiments may comprise a surfactant system comprising two or more surfactants. The surfactant system typically includes two or more surfactants selected from non-sulfate anionic surfactants, amphoteric surfactants, nonionic surfactants, and combinations of two or more thereof. For example, the surfactant system may include two, three, four, five, six, seven, eight, or nine or more surfactants. In some embodiments, the surfactant system comprises from two to eight, from two to seven, from two to six, from two to five, from two to four, from three to seven, from three to six, from three to five, from four to eight, four to seven, four to six, or any range or subrange thereof, of surfactants. Preferably, the surfactant system may include two or more surfactants, with the two or more surfactants being chosen from different categories of anionic surfactants, amphoteric surfactants, and nonionic surfactants. For example, the surfactant system may comprise at least one non-sulfate anionic surfactant, at least one amphoteric surfactant, and at least one nonionic surfactant. In certain embodiments, the surfactant system may comprise at least one sulfate-based anionic surfactant and at least one amphoteric surfactant.

In certain embodiments, the personal care composition comprises a sulfate-based surfactant system comprising at least one sulfate anionic surfactant and/or salt thereof. The at least one sulfate anionic surfactant and/or salt may, for example, comprise those selected from alkyl sulfates and alkyl ether sulfates. The alkyl sulfates and/or alkyl ether sulfates may have an alkyl group comprising from 8 to 20 carbon atoms, 8 to 16 carbon atoms, 10 to 16 carbon atoms, or 10 to 13 carbon atoms. Non-limiting examples of alkyl sulfate and alkyl ether sulfate salts include sodium lauryl sulfate (SLS), sodium dodecyl sulfate (SDS), sodium lauryl ether sulfate (SLES), ammonium lauryl sulfate, and ammonium laureth sulfate. In some embodiments, the sulfate anionic surfactants include sodium lauryl sulfate, sodium laureth sulfate, a salt thereof, or a combination of two or more thereof. In certain embodiments disclosed herein, the at least one sulfate anionic surfactant is SLES.

In certain embodiments, the personal care composition comprises from about 1 to about 26 wt. % of a surfactant system comprising at least one sulfate anionic surfactant, wherein the percentage is based on the total weight of the personal care composition. For example, the surfactant system comprising at least one sulfate anionic surfactant may be present in the personal care composition in an amount ranging from about 1 to about 23 wt. %, about 1 to about 20 wt. %, about 1 to about 18 wt. %, about 1 to about 16 wt. %, about 1 to about 14 wt. %, about 1 to about 12 wt. %, about 1 to about 10 wt. %; from about 3 to about 26 wt. %, about 3 to about 23 wt. %, about 3 to about 20 wt. %, about 3 to about 18 wt. %, about 3 to about 16 wt. %, about 3 to about 14 wt. %, about 3 to about 12 wt. %, about 3 to about 10 wt. %; from about 5 to about 26 wt. %, about 5 to about 23 wt. %, about 5 to about 20 wt. %, about 5 to about 18 wt. %, about 5 to about 16 wt. %, about 5 to about 14 wt. %, about 5 to about 12 wt. %, about 5 to about 10 wt. %; from about 7 to about 26 wt. %, about 7 to about 23 wt. %, about 7 to about 20 wt. %, about 7 to about 18 wt. %, about 7 to about 16 wt. %, about 7 to about 14 wt. %, about 7 to about 12 wt. %, about 7 to about 10 wt. %; from about 9 to about 26 wt. %, about 9 to about 23 wt. %, about 9 to about 20 wt. %, about 9 to about 18 wt. %, about 9 to about 16 wt. %, about 9 to about 14 wt. %, about 9 to about 12 wt. %; from about 11 to about 26 wt. %, about 11 to about 23 wt. %, about 11 to about 20 wt. %, about 11 to about 18 wt. %, about 11 to about 16 wt. %, about 11 to about 14 wt. %, or any range or subrange thereof, based on the total weight of the personal care composition. In certain embodiments, the surfactant system comprising at least one sulfate anionic surfactant may be present in the personal care composition in an amount ranging from about 8 to about 11 wt. %, such as from about 9 to about 10 wt. %, about 10 wt %, or about 9.8 wt %, based on the total weight of the personal care composition. In certain embodiments, the personal care composition comprises SLES as the sulfate anionic surfactant, and in certain embodiments, the SLES is present in the personal care composition in an amount ranging from about 8 to about 11 wt. %, such as from about 9 to about 10 wt. %, about 10 wt %, or about 9.8 wt %, based on the total weight of the personal care composition.

In some embodiments, the personal care compositions comprise a surfactant system that is essentially free of or free of sulfate-based anionic surfactants. For example, the personal care compositions may be formulated to have about 3 wt. % or less, about 2 wt. % or less, about 1 wt. % or less, about 0.5 wt. % or less, about 0.1 wt % or less, about 0.01 wt. % or less, or about 0.001 wt. % or less, of sulfate-based anionic surfactants, based on the total weight of the personal care composition, such as no detectable amount of a sulfate-based anionic surfactant. In certain embodiments, the personal care composition is free of sulfate-based anionic surfactants, such as free of SLES.

In certain embodiments wherein the personal care compositions comprise a surfactant system that is essentially free of or free of sulfate-based anionic surfactants, the surfactant system may comprise at least one non-sulfate based anionic surfactant. The non-sulfate based anionic surfactants may be selected from sulfonate surfactants, taurate surfactants, isethionate surfactants, sarcosinate surfactants, sulfonate surfactants, sulfosuccinate surfactants, sulfoacetate surfactants, alkoxylated monoacid surfactants, salts thereof, and combinations of two or more thereof. In certain embodiments, the non-sulfate based anionic surfactant is a long chain alkyl (e.g., C₆-C₂₂) anionic surfactant. In certain embodiments, the non-sulfate based anionic surfactant is disodium 2-sulfolaurate. The non-sulfate based anionic surfactant may have an alkyl and/or acyl group comprising 6 to 26 carbon atoms, 8 to 26 carbon atoms, 8 to 21 carbons, 8 to 16 carbon atoms, 10 to 16 carbon atoms, or 10 to 13 carbon atoms. In some instances, the non-sulfate based anionic surfactant comprises an alkyl and/or acyl group has more than 26 carbons.

The anionic surfactant may be selected from taurate surfactants having a structure according to the following formula:

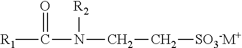

wherein R₁ is a saturated or unsaturated, straight or branched alkyl chain with 6 to 18 carbon atoms, R₂ is H or methyl, and M⁺ is H, sodium, or potassium (e.g., sodium methyl cocoyl taurate or sodium methyl oleoyl taurate).

Additional examples of taurate surfactants include sodium cocoyl taurate, potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations of two or more thereof. In some embodiments, the personal care composition comprises sodium lauroyl methyl taurate (or sodium methyl lauroyl taurate), sodium methyl cocoyl taurate (SMCT), or a combination thereof. In at least one preferred embodiment, the surfactant system comprises sodium methyl cocoyl taurate.

Non-limiting examples of isethionate surfactants include sodium isethionate, sodium cocoyl isethionate, sodium lauroyl methyl isethionate, and sodium cocoyl methyl isethionate. Sulfonated monoglycerides of fatty acids include sodium coconut monoglyceride sulfonates and the like. Examples of sarcosinate surfactants include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and combinations of two or more thereof. Non-limiting examples of alkyl taurate salts include sodium cocoyl taurate, sodium methyl cocoyl taurate, oleoyl taurate. In at least one embodiment, the personal care composition includes one or more anionic surfactant comprising oleoyl taurate and/or a salt thereof.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or a combination of two or more thereof.

Non-limiting examples of alkyl sulfacetates includes, e.g., alkyl sulfoacetates such as C₄-C₁₈ fatty alcohol sulfoacetates and/or salts thereof. A preferred sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

Examples of alkoxylated monoacids include, but are not limited to: butoxynol-5 carboxylic acid, butoxynol-19 carboxylic acid, capryleth-4 carboxylic acid, capryleth-6 carboxylic acid, capryleth-9 carboxylic acid, ceteareth-25 carboxylic acid, coceth-7 carboxylic acid, C₉₋₁₁ pareth-6 carboxylic acid, C₁₁₋₁₅ pareth-7 carboxylic acid, C₁₂₋₁₃ pareth-5 carboxylic acid, C₁₂₋₁₃ pareth-8 carboxylic acid, C₁₂₋₁₃ pareth-12 carboxylic acid, C₁₂₋₁₅ pareth-7 carboxylic acid, C₁₂₋₁₃ pareth-8 carboxylic acid, C₁₄₋₁₅ pareth-8 carboxylic acid, deceth-7 carboxylic acid, laureth-3 carboxylic acid, laureth-4 carboxylic acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, PPG-6-laureth-6 carboxylic acid, ppg-8-steareth-7 carboxylic acid, myreth-3 carboxylic acid, myreth-5 carboxylic acid, nonoxynol-5 carboxylic acid, nonoxynol-8 carboxylic acid, nonoxynol-10 carboxylic acid, octeth-3 carboxylic acid, octoxynol-20 carboxylic acid, oleth-3 carboxylic acid, oleth-6 carboxylic acid, oleth-10 carboxylic acid, ppg-3-deceth-2 carboxylic acid, capryleth-2 carboxylic acid, ceteth-13 carboxylic acid, deceth-2 carboxylic acid, hexeth-4 carboxylic acid, isosteareth-6 carboxylic acid, isosteareth-11 carboxylic acid, trudeceth-3 carboxylic acid, trideceth-6 carboxylic acid, trideceth-8 carboxylic acid, trideceth-12 carboxylic acid, trideceth-3 carboxylic acid, trideceth-4 carboxylic acid, trideceth-7 carboxylic acid, trideceth-15 carboxylic acid, trideceth-19 carboxylic acid, undeceth-5 carboxylic acid and a combination of two or more thereof.

The non-sulfate based anionic surfactant may be present in the personal care composition in an amount from about 0.3 to about 10 wt. %, based on the total weight of the personal care composition. For instance, the personal care composition may include non-sulfate based anionic surfactants in an amount ranging from about 0.3 to about 8 wt. %, about 0.3 to about 6 wt. %, about 0.3 to about 4 wt. %, about 0.3 to about 3 wt. %, about 0.3 to about 2 wt. %; from about 0.5 to about 10 wt. %, about 0.5 to about 8 wt. %, about 0.5 to about 6 wt. %, about 0.5 to about 4 wt. %, about 0.5 to about 3 wt. %, about 0.5 to about 2 wt. %; from about 1 to about 10 wt. %, about 1 to about 8 wt. %, about 1 to about 6 wt. %, about 1 to about 4 wt. %, about 1 to about 3 wt. %, about 1 to about 2 wt. %; from about 1.5 to about 10 wt. %, about 1.5 to about 8 wt. %, about 1.5 to about 6 wt. %, about 1.5 to about 4 wt. %, about 1.5 to about 3 wt. %; from about 2 to about 10 wt. %, about 2 to about 8 wt. %, about 2 to about 6 wt. %, about 2 to about 4 wt. %, about 2 to about 3 wt. %; from about 3 to about 10 wt. %, about 3 to about 8 wt. %, about 3 to about 6 wt. %, about 3 to about 4 wt. %; from about 4 to about 10 wt. %, about 4 to about 8 wt. %, about 4 to about 6 wt. %; from about 5 to about 10 wt. %, about 5 to about 8 wt. %, about 5 to about 6 wt. %, or any range or subrange thereof, based on the total weight of the personal care composition. In certain embodiments, the non-sulfate based anionic surfactant may be present in the personal care composition in an amount of about 1 wt. %, about 2 wt. %, 3 wt. %, about 4 wt. %, about 5 wt. %, or about 6 wt. %, based on the total weight of the personal care composition.

The surfactant system may further comprise one or more amphoteric surfactants. Amphoteric surfactants are typically characterized by a combination of high surfactant activity, lather forming and mildness. The amphoteric surfactant may comprise a substituent, such as an alkyl or acyl group, containing 8 to 18 carbon atoms and/or a substituent containing one or more carboxylate, sulfonate, sulfate, phosphate, or phosphonate. For instance, the amphoteric surfactant may have an alkyl group comprising from 8 to 20 carbon atoms, 8 to 16 carbon atoms, 10 to 16 carbon atoms, or 10 to 13 carbon atoms. The amphoteric surfactant(s) may include, but are not limited to, derivatives of aliphatic secondary and tertiary amines in which the aliphatic radical can be straight chain or branched and wherein one of the aliphatic substituents contains about 8 to about 18 carbon atoms and one contains an anionic water solubilizing group, e.g., carboxy, sulfonate, sulfate, phosphate, or phosphonate.

The amphoteric surfactants may include amphopropionate surfactants, betaine surfactants, sultaine surfactants, alkyl amphoacetate surfactants, and a combination of two or more thereof. In some instances, the personal care composition includes one or more alkyl amphopropionates. Non-limiting examples of alkyl amphopropionate surfactants include cocoamphopropionate, cornamphopropionate, caprylamphopropionate, cornamphopropionate, caproamphopropionate, oleoamphopropionate, isostearoamphopropionate, stearoamphopropionate, lauroamphopropionate, salts thereof, and a mixture thereof. In one embodiment, the alkyl amphopropionate surfactant comprises sodium cocoamphopropionate.

The personal care composition may include an amphoteric surfactant selected from betaine surfactants (also referred to as betaines). Examples of betaine surfactants include, but are not limited to high alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, and lauryl bis-(2-hydroxypropyl)alpha-carboxyethyl betaine; sulfobetaines, such as coco dimethyl sulfopropyl betaine, stearyl dimethyl sulfopropyl betaine; amido betaines; amidosulfobetaines; and the like. Preferably, a betaine surfactant is selected from coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and combinations of two or more thereof. In some embodiments, at least one betaine surfactant is selected from coco betaine, cocamidopropyl betaine, behenyl betaine, capryl/capramidopropyl betaine, and lauryl betaine, and a combination of two or more thereof. In further embodiments, the betaine surfactant is selected from cocamidopropyl betaine, lauryl dimethyl betaine, cetyl dimethyl betaine, and cocoamphodiacetate (e.g., disodium cocoamphodiacetate), and a combination of two or more thereof. In at least one embodiment, the personal care composition comprises disodium cocoamphodiacetate, cocamidopropyl betaine, or a combination thereof.

The amphoteric surfactant may, in some cases, be selected sultaines, also known as sulfobetaines, and hydroxy sultaines, for example, cocamidopropyl hydroxysultain. The sultaines may be an alkyl sultaines having from 6 to 20 carbons, 8 to 18 carbons, 8 to 16 carbons, or 10 to 16 carbons. Non-limiting examples of alkyl sultaines include hydroxyl sultaines having a structure according to the following formula:

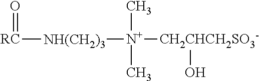

wherein R is an alkyl group having 8-18 carbon atoms. Examples of alkyl sultaine surfactants include, but are not limited to, cocamidopropyl hydroxysultaine, lauryl hydroxysultaine, and a combination of two or more thereof.

In some embodiments, the surfactant system includes an amphoteric surfactant selected from betaine surfactants and, optionally, an additional amphoteric surfactant other than betaines including those discussed above. The one or more betaine surfactants may, preferably, be selected from coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and a combination of two or more thereof.

In certain embodiments, the at least one amphoteric surfactant may be present in the personal care composition in an amount that may vary, but typically ranges from about 1 to about 26 wt. %, based on the total weight of the personal care composition. For example, the total amount of amphoteric surfactants in the personal care composition may be from about 1 to about 20 wt. %, about 1 to about 19 wt. %, about 1 to about 18 wt. %, about 1 to about 15 wt. %, about 1 to about 12 wt. %, about 5 to about 10 wt. %; from about 5 to about 20 wt. %, about 5 to about 19 wt. %, about 5 to about 18 wt. %, about 5 to about 15 wt. %, about 5 to about 12 wt. %; from about 10 to about 20 wt. %; from about 10 to about 19 wt. %, about 10 to about 18 wt. %, about 10 to about 15 wt. %, about 10 to about 12 wt. %; about 15 to about 20 wt. %; from about 16 to about 20 wt. %, about 17 to about 20 wt. %, about 18 to about 20 wt. %, about 18 to about 19 wt. %, about 19 to about 20 wt. %, or any range or subrange thereof, based on the total weight of the personal care composition. In certain embodiments, the one or more amphoteric surfactants is present in an amount ranging from about 1 to about 5 wt. %, such as about 3 to about 4 wt. %, based on the total weight of the personal care composition. In certain embodiments, the one or more amphoteric surfactants is present in an amount of about 12 wt. %, about 13 wt. %, about 14 wt. %, about 15 wt. %, about 16 wt. %, about 17 wt. %, about 18 wt. %, about 19 wt. %, or about 20 wt. %, based on the total weight of the personal care composition.

The surfactant system may comprise at least one nonionic surfactant. The at least one nonionic surfactant may be selected from glycoside surfactants, glucoside surfactants, glyceride surfactants, compounds produced by the condensation of alkylene oxide groups (hydrophilic in nature) with an organic hydrophobic compound, which may be aliphatic or alkylaromatic in nature, and combinations of two or more thereof. Preferably, at least one nonionic surfactant is selected from a glycoside surfactant, a glucoside surfactant, a glyceride surfactant, and a combination of two or more thereof. In some embodiments, the personal care composition comprises two or more (e.g., 2, 3, 4, or 5) nonionic surfactants.

The surfactant system may comprise at least one nonionic glyceride surfactant selected from glycerol monostarate, glyceryl oleate, glyceryl linoleate, glyceryl ricinoleate, PEG glyceryl oleate, PCA glyceryl oleate, and a combination of two or more thereof. In at least one embodiment, the PEG glyceryl oleate may be PEG-18 glyceryl oleate. Examples of glucoside surfactants include decyl glucoside, stearyl glucoside, lauryl glucoside, coco-glucoside, cetearyl glucoside, decyl lauryl glucoside, lauroyl ethyl glucoside, myristoyl ethyl glucoside, oleoyl ethyl glucoside, or a combination of two or more thereof. In some embodiments, the surfactant system includes one or more nonionic surfactant(s) chosen from lauryl glucoside, lauroyl ethyl glucoside, myristoyl ethyl glucoside, oleoyl ethyl glucoside, and a combination of two or more thereof. Additionally or alternatively, the glucoside surfactant may be chosen from polyglucosides, such as alkylpolyglucosides.

Further examples of nonionic surfactants include poloxamers, polyethylene oxide condensates of alkyl phenols, products derived from the condensation of ethylene oxide with the reaction product of propylene oxide and ethylene diamine, ethylene oxide condensates of aliphatic alcohols, acids, and esters, long chain tertiary amine oxides, long chain tertiary phosphine oxides, long chain dialkyl sulfoxides and mixtures of such materials. Additional examples of nonionic surfactants include polyoxyethylene, polyoxyethylene sorbitan esters, polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, or a combination of two or more thereof. In some instances, the nonionic surfactant comprises amine oxides, fatty acid amides, ethoxylated fatty alcohols, block copolymers of polyethylene glycol and polypropylene glycol, glycerol alkyl esters, polyoxyethytene glycol octylphenol ethers, sorbitan alkyl esters, polyoxyethylene glycol sorbitan alkyl esters, or a combination of two or more thereof.

In at least one embodiment, the nonionic surfactant comprises PEG methyl glucose dioleate. The PEG methyl glucose dioleate may be PEG-120 methyl glucose dioleate. In certain embodiments, the personal care compositions disclosed herein are free of or substantially free of PEG and any PEG-derived surfactants or thickeners.

The at least one nonionic surfactant may be present in an amount from about 1 to about 12 wt. %, based on the total weight of the personal care composition. In some instances, the at least one nonionic surfactant is present in the surfactant system in an amount from about 1 to about 10 wt. %, about 1 to about 8 wt. %, about 1 to about 6 wt. %, about 1 to about 5 wt. %, about 1 to about 4 wt. %; from about 2 to about 12 wt. %, about 2 to about 10 wt. %, about 2 to about 8 wt. %, about 2 to about 6 wt. %, about 2 to about 5 wt. %, about 2 to about 4 wt. %; from about 3 to about 12 wt. %, about 3 to about 12 wt. %, about 3 to about 10 wt. %, about 3 to about 8 wt. %, about 3 to about 6 wt. %, about 3 to about 5 wt. %, about 3 to about 4 wt. %; from about 4 to about 12 wt. %, about 4 to about 10 wt. %, about 4 to about 8 wt. %, about 4 to about 7 wt. %, about 4 to about 6 wt. %; from about 5 to about 12 wt. %, about 5 to about 10 wt. %, about 5 to about 8 wt. %, about 5 to about 7 wt. %; from about 6 to about 12 wt. %, about 6 to about 10 wt. %, about 6 to about 8 wt. %, about 7 to about 11 wt. %, or any range or subrange thereof, based on the total weight of the personal care composition.

The personal care composition may be formulated to have a surfactant system having certain weight ratios of the total amount of nonionic surfactant(s) to the total amount of amphoteric surfactant(s). For example, the surfactant system may have a weight ratio of the total amount of nonionic surfactant to amphoteric surfactant ranging from about 1.5:1 to about 7:1. In some embodiments, the personal care composition has a weight ratio of the total amount of nonionic surfactant to amphoteric surfactant ranging from about 1.5:1 to about 6:1, about 2:1 to about 6:1, about 3:1 to about 6:1, about 4:1 to about 6:1; from about 4.5:1 to about 6:1, about 4:1 to about 5:1, about 4.5:1, about 4.6:1, about 4.7:1, about 4.8:1, about 4.9:1, about 5:1, about 5.5:1, about 5.6:1, about 5.7:1, about 5.8:1, or about 5.9:1, or any range or subrange thereof.

The personal care composition may be formulated to have a surfactant system having certain weight ratios of the total amount of anionic surfactant(s) to the total amount of amphoteric surfactant(s). For example, in some preferred embodiments, the personal care composition has a weight ratio of non-sulfate based anionic surfactant(s) to amphoteric surfactant(s) ranging from about 2:1 to about 1:9, about 1:2 to about 1:8, about 1:2 to about 1:7, about 1:2 to about 1:6.5, about 1:2 to about 1:6; from about 1:3 to about 1:9, about 1:3 to about 1:8, about 1:3 to about 1:7, about 1:3 to about 1:6.5, about 1:3 to about 1:6; from about 1:3.5 to about 1:9, about 1:3.5 to about 1:8, about 1:3.5 to about 1:7, about 1:3.5 to about 1:6.5, about 1:3.5 to about 1:6; about 1:4 to about 1:9, about 1:4 to about 1:8, about 1:4 to about 1:7, about 1:4 to about 1:6.5, about 1:4 to about 1:6, or any range or subrange thereof.

The personal care compositions disclosed herein may further comprise at least one tear film protecting agent. As used herein, the term “tear film protecting agent” indicates an ingredient that may reduce irritation to the eyes. In certain embodiments, the at least one tear film protecting agent is chosen from ingredients that are mild and gentle on the eyes, including ingredients that are all natural. The human eye contains a thin liquid layer that covers the outer surfaces of the eye, known as the tear film. The tear film is approximately 3 μL of fluid forming a layer approximately 3 μm thick. The tear film contains two layers, an aqueous layer and a lipid layer. While the lipid layer serves to prevent evaporation of tears from the surface of the eye, the aqueous layer is responsible for the forming the majority of tears, which serve to provide oxygen and nutrients to the eyes, as well as to remove unwanted debris. In certain embodiments, the personal care composition comprises at least two tear film protecting agents, such as, for example, at least one hydrophilic tear film protecting agent that serves to protect the aqueous layer of the tear film and at least one hydrophobic tear film protecting agent that serves to protect the lipid layer of the tear film.

In certain embodiments, the at least one tear film protecting agent is hydrophilic and comprises a polyol. The polyol(s) may be chosen from glycols or compounds with numerous hydroxyl groups. In some cases, the one or more polyols is/are selected from the group consisting of C₂-C₃₂ polyols. The one or more polyols may be liquid at ambient temperature (25° C.). The one or more polyols may have from 2 to 32 carbon atoms, from 3 to 16 carbon atoms, or from 3 to 12 carbon atoms. Polyols that may be included in the personal care composition, in certain instances, include ethylene glycol, propylene glycol, butylene glycol, hexylene glycol, glycerin, diglycerin, diethylene glycol, and dipropylene glycol, and mixtures thereof. In some cases, the personal care composition comprises glycerin, and optionally one or more polyols other than glycerin. Non-limiting examples of polyols that may, optionally, be included in the personal care include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture thereof.

In certain embodiments the at least one tear film protecting agent is hydrophilic, and in certain embodiments, the at least one tear film protecting agent is glycerin. In certain embodiments, the at least one hydrophilic tear film protecting agent, such as glycerin, is present in the personal care composition in an amount ranging from about 1 to about 30 wt. %, based on the total weight of the personal care composition. For example, the total amount of polyol(s) in the personal care composition may be from about 1 to about 25 wt. %, about 1 to about 22 wt. %, about 1 to about 18 wt. %, about 1 to about 12 wt. %, about 1 to about 8 wt. %, about 1 to about 6 wt. %, about 1 to about 5 wt. %, about 1 to about 4 wt. %, about 1 to about 3 wt. %; from about 2 to about 30 wt. %, about 2 to about 25 wt. %, about 2 to about 22 wt. %, about 2 to about 18 wt. %, about 2 to about 12 wt. %, about 2 to about 8 wt. %, about 2 to about 6 wt. %, about 2 to about 5 wt. %, about 2 to about 4 wt. %, about 2 to about 3 wt. %; from about 3 to about 22 wt. %, about 3 to about 18 wt. %, about 3 to about 12 wt. %, about 3 to about 8 wt. %, about 3 to about 6 wt. % about 3 to about 5 wt. %; from about 5 to about 25 wt. %, about 5 to about 22 wt. %, about 5 to about 20 wt. %, about 5 to about 18 wt. %, about 5 to about 16 wt. %, about 5 to about 12 wt. %, about 5 to about 10 wt. %, about 5 to about 8 wt. %; from about 7 to about 30 wt. %, about 7 to about 25 wt. %, about 7 to about 22 wt. %, about 7 to about 20 wt. %, about 7 to about 18 wt. %, about 7 to about 16 wt. %; from about 9 to about 30 wt. %, about 9 to about 25 wt. %, about 9 to about 22 wt. %, about 9 to about 20 wt. %, about 9 to about 18 wt. %, about 9 to about 16 wt. %; from about 12 to about 30 wt. %, about 12 to about 25 wt. %, about 12 to about 22 wt. %, about 12 to about 20 wt. %, about 12 to about 18 wt. %, about 12 to about 16 wt. %; from about 14 to about 30 wt. %, about 14 to about 25 wt. %, about 14 to about 22 wt. %, about 14 to about 20 wt. %, about 14 to about 18 wt. %; from about 16 to about 30 wt. %, about 16 to about 25 wt. %, about 16 to about 22 wt. %, about 16 to about 20 wt. %; from about 18 to about 30 wt. %, about 18 to about 25 wt. %, about 21 to about 25 wt. %, or any range or subrange thereof, based on the total weight of the personal care composition.

In certain embodiments, the at least one tear film protecting agent may be a hydrophobic agent. In certain embodiments, the at least one hydrophobic tear film protecting agent is selected from glyceride surfactants, such as glycerol monostarate, glyceryl oleate, glyceryl linoleate, glyceryl ricinoleate, PEG glyceryl oleate, PCA glyceryl oleate, and a combination of two or more thereof. In certain embodiments, the at least one tear film protecting agent is glyceryl oleate. In certain embodiments, the glyceryl oleate may be added to the composition together as part of another ingredient or mixture of ingredients, such as in form of an ingredient in Lamesoft® PO-65, available from BASF.

In certain embodiments, the at least one hydrophobic tear film protecting agent may be present in the personal care composition in an amount ranging from about 0.1 to about 7 wt. %, such as about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 75 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %, or any range or subrange thereof, based on the total weight of the personal care composition.

In certain embodiments, the personal care composition comprises at least one hydrophilic tear film protecting agent (e.g., glycerin) and at least one hydrophobic tear film protecting agent (e.g., glyceryl oleate).

The personal care composition may optionally include at least one thickening agent, and in certain embodiments, the personal care composition may be essentially free of or free of at least one thickening agent, wherein the thickening agent is not part of the surfactant system. The amount of thickening agent in the personal care composition, if present, may be from about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %, or any range or subrange thereof, based on the total weight of the personal care composition.

Without being limited to any particular theory, the personal care compositions in some embodiments achieve preferable viscosities with a minimal amount or while being essentially free of or free of thickening agents (e.g., thickening agents other than surfactants). For example, the personal care composition may include thickening agent(s) in a total amount from about 0.1 wt. %, from about 0.5 wt. %, from about 1 wt. %, from about 2 wt. %, from about 3 wt. %, or from about 3.5 wt. % up to about 1 wt. %, up to about 2 wt. %, up to about 3 wt. %, up to about 4 wt. %, or up to about 5 wt. %, including any range or subrange formed therefrom, based on the total weight of the personal care composition. In some embodiments, the personal care composition may have thickening agent(s) in a total amount of about 5 wt. % or less, about 4 wt. % or less, about 3 wt. % or less, about 2 wt. % or less, about 1 wt. % or less, about 0.5 wt. % or less, or about 0.1 wt. % or less, based on the total weight of the personal care composition. In some preferred embodiments, the personal care composition is free of thickening agents except for those constituting surfactants.

Thickening agents may be referred to as “thickeners” or “viscosity modifying agents.” Thickening agents are typically included to increase the viscosity of the personal care compositions. Non-limiting examples of thickening agents that may be incorporated into or excluded from the personal care compositions include natural gums, polyacrylate crosspolymers or cross linked polyacrylate polymers, cationic acrylate copolymers, anionic acrylic or carboxylic acid polymers, polyacrylamide polymers, polysaccharides such as cellulose derivatives, cationic gums, polyquaterniums, vinylpyrrolidone homopolymers/copolymers, C₈₋₂₄ hydroxyl substituted aliphatic acid, C₈₋₂₄ conjugated aliphatic acid, sugar fatty esters, polyglyceryl esters, and a mixture thereof. Particular types of thickening agents that may be mentioned include the following: gums (e.g., natural gums, xanthum gums), sucrose esters, polyvinylpyrrolidone (PVP) and co-polymers, celluloses, polyquaternium compounds, and carboxylic acid or carboxylate based homopolymer or co-polymer, which can be linear or crosslinked.

In some embodiments, the personal care compositions are essentially free of or free of acrylate based thickener(s). For example, the personal care compositions may be formulated to have about 6 wt. % or less, about 4 wt. % or less, about 3 wt. % or less, about 2 wt. % or less, about 1 wt. % or less, about 0.5 wt. % or less, or about 0.1 wt. % or less, of acrylate based thickener, based on the total weight of the personal care composition. The personal care composition is free of acrylate based thickener in at least one embodiment.

Additionally or alternatively, the personal care compositions may be essentially free of or free of polymer based thickener(s). For example, the personal care compositions may be formulated to have about 6 wt. % or less, about 4 wt. % or less, about 3 wt. % or less, about 2 wt. % or less, about 1 wt. % or less, about 0.5 wt. % or less, or about 0.1 wt. % or less, of polymer based thickener, based on the total weight of the personal care composition. The personal care composition is free of polymer based thickener in at least one embodiment.

The personal care compositions may also be essentially free of or free of polysaccharide based thickener(s). For example, the personal care compositions may be formulated to have about 6 wt. % or less, about 4 wt. % or less, about 3 wt. % or less, about 2 wt. % or less, about 1 wt. % or less, about 0.5 wt. % or less, or about 0.1 wt. % or less, of polysaccharide based thickener, based on the total weight of the personal care composition. The personal care composition is free of polysaccharide based thickener in at least one embodiment.

Additionally or alternatively, the personal care composition may be essentially free of or free of thickener(s) (e.g., other than those constituting surfactants). For example, the personal care compositions may be formulated to have about 6 wt. % or less, about 4 wt. % or less, about 3 wt. % or less, about 2 wt. % or less, about 1 wt. % or less, about 0.5 wt. % or less, or about 0.1 wt. % or less, of thickener(s) (e.g., other than those constituting surfactants), based on the total weight of the personal care composition. The personal care composition is free of thickeners in at least one embodiment.

In certain embodiments, the personal care compositions disclosed herein may further comprise at least one anti-irritant and/or at least one anti-allergy agent. Exemplary anti-irritants or anti-allergy agents may include, for example, dipropylene glycol (DPG), methyl salicylate, capsaicin, eugenol, menthol, oil of wintergreen, camphor, eucalyptus, eucalyptol, mustard plaster, turpentine oil, safrol, menthyl lactate, menthyl ethoxyacetate, menthone glycerinacetal, 3-I-menthoxypropane-1,2-diol, ethyl I-menthyl carbonate, (1 S,3S,4R)-p-menth-8-en-3-ol, menthyl pyrrolidone carboxylate, N-substituted-p-menthane-3-carboxamides, ketal coolants including, for example, I-menthon-Id-isomenthon glycerin ketal. Other examples of agents with soothing properties that may encompass at least one anti-irritant or at least one anti-allergy agent include, but are not limited to herb extracts, such as, aloe vera, alpha-bisabolol, D-panthenol, allantoin, hamamelis, chamomile, yarrow; vitamins, such as, tocopheryl acetate (vitamin E); calendula, comfrey, witch hazel and other astringents, sea weed, and oat extracts; oils, such as, almond oil, avocado oil, and comfrey; and essential oils, such as, cardamone, eucalyptus, Mentha piperita (peppermint), hyssop, and rosemary; waxy or unctuous substances such as lanolin or vaseline jelly, minerals, such as, zinc oxide, calamine and selenium, and combinations thereof.

The at least one anti-irritant and/or at least one anti-allergy agent may be present in the personal care composition in any amount sufficient to provide an anti-irritant and/or anti-allergy effect to the user, such as, for example, present in an amount ranging from about 0.1 to about 5 wt. %, based on the total weight of the personal care composition. For instance, the personal care composition may be formulated to have at least one anti-irritant and/or at least one anti-allergy agent present in an amount from about 0.1 to about 4 wt. %, about 0.1 to about 3 wt. %, about 0.1 to about 2 wt. %; about 1 to about 4 wt. %, about 1 to about 3 wt. %, about 1 to about 2 wt. %; from about 2 to about 4 wt. %, about 2 to about 3 wt. %; about 3 to about 4 wt. %, or any range or subrange thereof, based on the total weight of the personal care composition.

In certain embodiments, the personal care composition include salt in an amount ranging from about 0.1 to about 4.5 wt. %, based on the total weight of the personal care composition. For instance, the personal care composition may be formulated to have salt present in an amount from about 0.1 to about 4.5 wt. %, about 0.1 to about 4 wt. %, about 0.1 to about 3.5 wt. %, about 0.1 to about 3 wt. %, about 0.1 to about 2.5 wt. %, about 0.1 to about 2 wt. %; from about 0.5 to about 4.5 wt. %, about 0.5 to about 4 wt. %, about 0.5 to about 3.5 wt. %, about 0.5 to about 3 wt. %, about 0.5 to about 2.5 wt. %, about 0.5 to about 2 wt. %; from about 1 to about 4.5 wt. %, about 1 to about 4 wt. %, about 1 to about 3.5 wt. %, about 1 to about 3 wt. %, about 1 to about 2.5 wt. %, about 1 to about 2 wt. %; from about 1.5 to about 4.5 wt. %, about 1.5 to about 4 wt. %, about 1.5 to about 3.5 wt. %, about 1.5 to about 3 wt. %, about 1.5 to about 2.5 wt. %; from about 2 to about 4.5 wt. %, about 2 to about 4 wt. %, about 2 to about 3.5 wt. %, about 2 to about 3 wt. %; from about 2.5 to about 4.5 wt. %, about 2.5 to about 4 wt. %, about 2.5 to about 3.5 wt. %; from about 3 to about 4.5 wt. %, about 3 to about 4 wt. %, or any range or subrange thereof, based on the total weight of the personal care composition. The salt may be a an inorganic salt, such as sodium chloride, sodium sulfate, calcium chloride, potassium chloride, or a combination thereof.

The personal care compositions may be formulated to have a viscosity of about 3,000 to about 15,000 cP using a Brookfield DV2T viscometer with a spindle number 5 at 20 RPM and a temperature of 25° C. in a non-diluted form. In some instances, the personal care composition has a viscosity of from about 3,000 to about 15,000 cP, about 3,000 to about 12,000 cP, about 3,000 to about 10,000 cP, about 3,000 to about 8,000 cP, about 3,000 to about 7,000 cP, about 3,000 to about 6,000 cP; from about 3,500 to about 15,000 cP, about 3,500 to about 12,000 cP, about 3,500 to about 10,000 cP, about 3,500 to about 8,000 cP, about 3,500 to about 7,000 cP, about 3,500 to about 6,000 cP; from about 4,000 to about 15,000 cP, about 4,000 to about 12,000 cP, about 4,000 to about 10,000 cP, about 4,000 to about 8,000 cP, about 4,000 to about 7,000 cP, about 4,000 to about 6,000 cP; from about 4,500 to about 15,000 cP, about 4,500 to about 12,000 cP, about 4,500 to about 10,000 cP, about 4,500 to about 8,000 cP, about 4,500 to about 7,000 cP, about 4,500 to about 6,000 cP, or any range or subrange thereof using a Brookfield DV2T viscometer with a spindle number 5 at 20 RPM and a temperature of 25° C.

The personal care composition generally comprises water. Water may be present in the personal care composition in an amount ranging from about 45 to about 94 wt. %, based on the total weight of the personal care composition. For example, the personal care composition may comprise water in an amount from about 55 to about 94 wt. %, about 65 to about 94 wt. %, about 68 to about 94 wt. %, about 71 to about 94 wt. %, about 75 to about 94 wt. %, about 78 to about 94 wt. %, about 81 to about 94 wt. %, about 84 to about 94 wt. %; from about 45 to about 91 wt. %, about 55 to about 91 wt. %, about 65 to about 91 wt. %, about 68 to about 91 wt. %, about 71 to about 91 wt. %, about 75 to about 91 wt. %, about 78 to about 91 wt. %, about 81 to about 91 wt. %; from about 45 to about 88 wt. %, about 55 to about 88 wt. %, about 65 to about 88 wt. %, about 68 to about 88 wt. %, about 71 to about 88 wt. %, about 75 to about 88 wt. %, about 78 to about 88 wt. %; from about 45 to about 85 wt. %, about 55 to about 85 wt. %, about 65 to about 85 wt. %, about 68 to about 85 wt. %, about 71 to about 85 wt. %, about 75 to about 85 wt. %, about 78 to about 85 wt. %; from about 45 to about 82 wt. %, about 55 to about 82 wt. %, about 65 to about 82 wt. %, about 68 to about 82 wt. %, about 71 to about 82 wt. %, about 75 to about 82 wt. %; from about 45 to about 79 wt. %, about 55 to about 79 wt. %, about 65 to about 79 wt. %, about 68 to about 79 wt. %; from about 45 to about 76 wt. %, about 55 to about 76 wt. %, about 65 to about 76 wt. %, about 68 to about 76 wt. %, or any range or subrange thereof, based on the total weight of the personal care composition.

The personal care compositions disclosed herein may be slightly acidic. For example, in certain embodiments, the personal care compositions disclosed herein have a pH ranging from 2 to 7, such as 3 to 6, or 4 to 5, or any range or subrange thereof. In certain embodiments, the personal care compositions disclosed herein have a pH greater than 3, such as a pH of 3.2, a pH of 3.5, a pH of 4, a pH of 4.2, a pH of 4.5, a pH of 5, a pH of 5.2, or a pH of 5.5.

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, lactic acid and the like. The personal care composition may include one or more bases, such as sodium hydroxide, potassium hydroxide and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final form of personal care composition and/or product (e.g., in a non-diluted/concentrated form and/or in a diluted/ready-to-use form). For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, about 0.1 to about 1 wt. %, about 0.2 to about 0.5 wt. %, including ranges and sub-ranges therebetween, based on the total weight of the personal care composition.

The personal care compositions typically comprise a preservative system. The preservative system may comprise lactic acid, citric acid, sodium benzoate, potassium sorbate, salicylic acid, tetrasodium N,N-bis(carboxymethyl)-L-glutamate, a salt thereof or a combination of two or more thereof. The preservative system may include sodium lactate, sodium salicylate, calcium lactate, and/or sodium citrate. In some embodiments, the preservative system comprises citric acid and/or a salt thereof. In further embodiments, the preservative system comprises tetrasodium N,N-bis(carboxymethyl)-L-glutamate and at least one preservative selected from potassium sorbate, sodium benzoate, and a combination of two or more thereof. Additionally or alternatively, the preservative system may include at least one of lactic acid, tetrasodium N,N-bis(carboxymethyl)-L-glutamate and at least one preservative selected from potassium sorbate, sodium benzoate, and a combination of two or more thereof.

The personal care composition may further comprise one or more colorants. The colorants may be a pigment, a dye, or mixtures thereof. Non-limiting examples of pigments include titanium dioxide, zinc oxide, kaolin, mica etc. Non-limiting examples of dyes include food dyes suitable for food, drug and cosmetic applications, and mixtures thereof. Some color agents (colorants) are known as FD&C dyes. In some embodiments, the colorants may be present in an amount ranging from about 0.0001 to about 0.4 wt. %, including all percentages and subranges therebetween, based on the total weight of the personal care composition. In further embodiments, the colorants may be present in an amount ranging from about 0.0001 to about 4 wt. %, including all percentages and subranges therebetween, based on the total weight of the personal care composition.

The personal care compositions disclosed herein may be manufactured using methods known in the art. Typically, the ingredients are combined and optionally heated where components need to be melted. The components are mixed. Desirably, volatile materials such as fragrant materials are incorporated in the composition in the latter stages of a mixing cycle in order to avoid volatilization thereof. After mixing, the composition may be poured directly into the dispensers and the container capped to preserve the product until use.

In another aspect, the disclosure provides a method of depositing a personal care composition as disclosed herein on the skin and/or hair, comprising applying an effective amount of the personal care compositions disclosed herein to the skin and/or hair. The method further comprises optionally lathering the personal care composition, e.g., by creating a foam with or without the presence of water, and then optionally rinsing the personal care composition from the skin and/or hair, e.g., with water.

### EXAMPLES

### Example 1

Four non-limiting, example personal care compositions (Example Compositions A, B, C, and D) were prepared and evaluated against a comparative formulation that is child's 3-in-1 shower gel product advertised as being a “no tears” formulation.

Composition A contained an SLES and cocoamidopropyl betaine surfactant system. Compositions B-D were free of SLES and contained the following surfactant systems:
Composition B: cocoamidopropyl betaine, coco-glucoside, disodium cocoamphodiacetate (3.95%), and disodium-2-sulfolaurate;
Composition C: cocoamidopropyl betaine, coco-glucoside, sodium methyl oleoyl taurate, and disodium-2-sulfolaurate; and
Composition D: cocoamidopropyl betaine, coco-glucoside, disodium cocoamphodiacetate (6.565%), and disodium-2-sulfolaurate.

The tear film protection used in Composition A was 5% glycerin, while the tear film protection used in Compositions B-C was 5% glycerin and 0.75% glyceryl oleate.

The formulations of Example Compositions A-D are shown in Table 1 below.

TABLE 1
Summary of Test Compositions

| | SLES | | | |
|---|---|---|---|---|
| | Backbone | Sulfate-free Backbone | | |
| | Ex. A | Ex. B | Ex. C | Ex. D |
| Ingredient | (wt. %) | (wt. %) | (wt. %) | (wt. %) |
| Demineralized water | QS | QS | QS | QS |
| SLES | 9.8 | — | — | — |
| Cocamidopropyl betaine | 4.9 | 15.0 | 18.0 | 12.0 |
| Trisodium ethylenediamine | 0.05 | — | — | — |
| disuccinate | | | | |
| Glycerin | 5.0 | 5.0 | 5.0 | 5.0 |
| Sodium benzoate | 0.5 | 0.28 | 0.28 | 0.28 |
| Citric acid | 0.52 | — | — | — |
| Sodium chloride | 1.0 | — | — | — |
| N,N-Bis(carboxymethyl)-L- | — | 0.21 | 0.21 | 0.21 |
| glutamic acid tetrasodium salt | | | | |
| Potassium sorbate | — | 0.40 | 0.40 | 0.40 |
| Disodium-2-sulfolaurate | — | 4.26 | 5.14 | 3.12 |
| Lamesoft ® PO-65 | — | 2.5 | 2.5 | 2.5 |
| (cocoglucoside/glyceryl | | | | |
| oleate mixture) | | | | |
| Disodium | — | 3.95 | — | 6.58 |
| Cocoamphodiacetate | | | | |
| Cocoglucoside | — | 1.5 | 2.1 | 1.4 |
| Lactic acid | — | 0.2 | 0.2 | 0.2 |
| Sodium Methyl Oleoyl | — | — | 1.0 | — |
| Taurate | | | | |
| % Biodegradability | 99.53 | 100 | 97 | 100 |
| % Natural Origin Index | 97.22 | 96.74 | 96.88 | 96.76 |
| Target pH | 4.2 | 5.2 | 5.2 | 5.2 |
| Surfactant level % | 8.3 | 9.2 | 9.3 | 8.8 |

Example Compositions A-D were evaluated for stability in 13-week accelerated aging conditions. The stability study results are shown below in Table 2.

TABLE 2
Formula Stability Studies

| | | Aged Result - 3 mo | Aged Result - 3 mo |
|---|---|---|---|
| Composition | Initial Result | (40° C./75% RH) | (4° C.) |
| Composition | pH: 4.22 | pH: 4.32 | pH: 4.31 |
| A | Viscosity: | Viscosity: | Viscosity: |
| | 4640 cps | 5860 cps | 4820 cps |
| Composition | pH: 5.20 | pH: 5.08 | pH: 5.27 |
| B | Viscosity: | Viscosity: | Viscosity |
| | 9680 cps | 9580 | 11080 cps |
| Composition | pH: 5.20 | pH: 5.22 | pH: 5.26 |
| C | Viscosity: | Viscosity: | Viscosity: |
| | 13660 cps | 14000 cps | 14080 cps |
| Composition | pH: 5.22 | pH: 5.19 | pH: 5.23 |
| D | Viscosity: | Viscosity: | Viscosity: |
| | 5120 cps | 15080 cps | 11620 cps |

Hand wash and SITA FoamTester foam studies were conducted, using a comparative product (Comparative #1) and a shower gel product (Comparative #2) as benchmarks. The Comparative #1 contained the following ingredients: water; a surfactant system comprising cocamidopropyl betaine, sodium myreth sulfate, and decyl glucoside; sodium benzoate; glycerin; bisabolol; and a thickening system comprising PEG-200 hydrogenated glyceryl palmate, guar hydroxypropyltrimonium chloride, and sodium chloride. The comparative product #1 had a pH of about 4.5 and a viscosity (sp #4, 20 rpm, 1 min) of about 3700 cps. Comparative Product #2 contained decyl glucoside and xanthum gum.

In the hand wash test Composition B performed the best, followed by, in order, Composition C, Composition A, Comparative #1, Composition D, and Comparative #2. In the SITA foam test, foam volume and bubble size were evaluated. As for foam volume, Composition A had the highest foam volume, followed by Composition B and Composition C, all of which outperformed Composition D, Comparative #1, and Comparative #2. Comparative #1 exhibited a foam volume/mL of 835.5 and Comparative #2 exhibited a foam volume/mL of 647.6. Compositions A, B, and C had foam volume/mL of 873.6, 857.7, and 856.1, respectively, and Composition D had a foam volume/mL of 621.5.

### Example 2—Human Eye Irritation (HEI) Testing

HEI testing was performed on each of the Compositions A-D, as well as Comparative #1. Prior to HEI testing and at the conclusion of testing, the eyes of all subjects were examined and graded by an ophthalmologist for the presence or absence of tearing, conjunctival irritation, and overall redness or bloodshot appearance of the sclera. The parameters were evaluated on a scale of 0 to 4 as follows: (0) none; (0.5) very mild; (1) mild; (2) moderate; (3) marked; and (4) very severe. A visual acuity test was performed on each subject prior to and following the administration of drops and evaluation. Additionally, subjects were asked to score the intensity or perceived discomfort of the drops within 10 seconds of administration using the following Perceived Discomfort Scale of 0-5: (0) no sting; (1) very slight sting; (2) mild sting, no pain; (3) moderate sting, with or without pain; (4) bearable pain and stinging; and (5) severe pain and unbearable stinging.

Drops of the formulations (45 μL) were placed into 20 subjects' eyes. For each subject, control drops were placed in one eye (either left or right), and test drops were placed in the alternate eye.

For Phase I HEI testing, a control and the test formulations were diluted into different four solutions ranging from 1-7.5% (1%, 3%, 5%, and 7.5%, n=5 for all groups). Next, for Phase II HEI testing, the control and test formulations were diluted into 7.5% solutions, and drops (45 μL) were administered to subjects, and the results were graded by an ophthalmologist.

In the HEI test, Composition A was associated with more perceived discomfort, conjunctival irritation, scleral redness, and tearing than Comparative #1. Nonetheless, as shown below in Table 3, the results were not significant.

TABLE 3
HEI Testing Results for Composition A (SLES Formulation)

| Category Evaluated | Comp. A | Comp. #1 | p-value |
|---|---|---|---|
| Perceived discomfort/sting | 1.55 | 1.40 | 0.694 |
| Conjunctival irritation | 0.38 | 0.30 | 0.813 |
| Scleral redness | 0.38 | 0.28 | 0.688 |
| Tearing | 0.03 | 0.00 | 1.000 |
| Visual acuity | No change | No change | — |

HEI testing was further performed on the sulfate-free formulations of Compositions B-D, which contained a surfactant system comprising cocamidopropyl betaine, coco-glucoside, disodium-2-sulforlaurate, and sodium methyl oleoyl taurate or sodium cocoamphodiacetate, as disclosed above. The results are shown below in Tables 4-6.

TABLE 4
HEI Testing Results for Composition B

| Category Evaluated | Comp. B | Comp. #1 | p-value |
|---|---|---|---|
| Perceived discomfort/sting | 0.60 | 1.40 | 0.012 |
| Conjunctival irritation | 0.10 | 0.45 | 0.023 |
| Scleral redness | 0.10 | 0.45 | 0.023 |
| Tearing | 0.00 | 0.00 | 1.000 |
| Visual acuity | No change | No change | — |

TABLE 5
HEI Testing Results for Composition C

| Category Evaluated | Comp. C | Comp. #1 | p-value |
|---|---|---|---|
| Perceived discomfort/sting | 0.65 | 1.30 | 0.014 |
| Conjunctival irritation | 0.15 | 0.45 | 0.041 |
| Scleral redness | 0.15 | 0.45 | 0.041 |
| Tearing | 0.00 | 0.00 | 1.000 |
| Visual acuity | No change | No change | — |

TABLE 6
HEI Testing Results for Composition D

| Category Evaluated | Comp. D | Comp. #1 | p-value |
|---|---|---|---|
| Perceived discomfort/sting | 0.80 | 1.40 | 0.034 |
| Conjunctival irritation | 0.30 | 0.53 | 0.063 |
| Scleral redness | 0.30 | 0.58 | 0.063 |
| Tearing | 0.00 | 0.03 | 1.000 |
| Visual acuity | No change | No change | — |

As shown above, all four test Compositions A-D passed the HEI testing. Overall, the three sulfate-free formulations performed better than the SLES formulation. While not wishing to be bound by theory, it is believed this may be due to a milder surfactant system, higher pH, and more tear film protectant. While not wishing to be bound by theory, the superior performance of Composition B and C relative to Composition D may be due to the higher level of disodium cocoamphodiacetate in Composition D.

The present disclosure has been described with reference to exemplary embodiments. Although a limited number of embodiments have been shown and described, it will be appreciated by those skilled in the art that changes may be made in these embodiments without departing from the principles and spirit of the preceding detailed description. It is intended that the present disclosure be construed as including all such modifications and alterations insofar as they come within the scope of the appended claims or the equivalents thereof.

What is claimed is:

1. A personal care composition comprising:
(a) a surfactant system comprising at least one anionic surfactant and at least one amphoteric surfactant; and
(b) at least one tear film protecting agent,
wherein the personal care composition has a pH ranging from about 4.0 to about 6.0.

2. The personal care composition according to claim 1, wherein the personal care composition has a natural origin index of about 90% or more, based on ISO Standard 16128.

3. The personal care composition according to claim 2, wherein the natural origin of the personal care composition is about 96% or more, based on ISO Standard 16128.

4. The personal care composition according to claim 1, wherein the at least one anionic surfactant is selected from sulfate anionic surfactants.

5. The personal care composition according to claim 4, wherein the sulfate anionic surfactant is sodium laureth sulfate (SLES).

6. The personal care composition according to claim 1, wherein the at least one anionic surfactant is selected from sulfolaurate surfactants.

7. The personal care composition according to claim 6, wherein the sulfolaurate surfactant is disodium-2-sulfolaurate.

8. The personal care composition according to claim 1, wherein the at least one amphoteric surfactant is selected from betaine surfactants.

9. The personal care composition according to claim 8, wherein the betaine surfactant is cocamidopropyl betaine.

10. The personal care composition according to claim 1, wherein the surfactant system further comprises at least one non-ionic surfactant.

11. The personal care composition according to claim 10, wherein the at least one non-ionic surfactant is selected from glucoside surfactants.

12. The personal care composition according to claim 1, wherein the at least one tear film protecting agent is selected from glycerin and glyceryl oleate.

13. The personal care composition according to claim 1, wherein the surfactant system further comprises a taurate surfactant.

14. The personal care composition according to claim 1, wherein the pH ranges from about 4.2 or about 5.2.

15. The personal care composition according to claim 1, further comprising at least one preservative selected from sodium benzoate and potassium sorbate.

16. The personal care composition according to claim 1, wherein the personal care composition is free of sulfate anionic surfactants.

17. The personal care composition according to claim 1, further comprising at least one anti-allergy agent.

18. The personal care composition according to claim 1, wherein the at least one tear-protecting agent is present in an amount from about 4 to about 8 wt. %, based on the total weight of the personal care composition.

19. The personal care composition according to claim 1, wherein the personal care composition is free of thickeners.

20. The personal care composition according to claim 1, wherein the at least one anionic surfactant is present in an amount from about 3 to about 10 wt. %, based on the total weight of the personal care composition.
